(19) United States
(12) Patent Application Publication
(10) Pub. No.: US 2019/0339771 A1
(43) Pub. Date: Nov. 7, 2019

(54) Method, System and Apparatus For Brainwave and View Based Recommendations and Story Telling

(71) Applicant: Sameer Yami, Milpitas, CA (US)

(72) Inventor: Sameer Yami, Milpitas, CA (US)

(21) Appl. No.: 15/971,989

(22) Filed: May 4, 2018

(51) Int. Cl. G06F 3/01; A61B 5/04; A61B 5/0482; G06T 19/00; A61B 5/16; H04N 21/8541

(52) U.S. Cl. CPC G06F 3/015; A61B 5/04012; A61B 5/0482; H04N 21/8545; A61B 5/165; H04N 21/8541; G06T 19/006

(57) ABSTRACT

A method, system and apparatus for gathering sensor data, identifying physical objects in a spatial location, detecting brain activity of humans in that location, using the brain activity to create user specific or combined models, and recommending this location, alternative stories, and the humans in this location to an end user based on end user's brain activity-based preferences, and end user's viewing angle.

Based on Provisional Application No. 62/502,114

(A METHOD, SYSTEM AND APPARATUS FOR BRAINWAVE AND VIEW BASED RECOMMENDATIONS AND STORYTELLING)

### TECHNICAL FIELD

The present invention relates generally to computer software systems. In particular, an embodiment of the invention relates to a method, system and apparatus detecting physical objects and people's brain activities and angle of viewing using sensor data and making recommendations based on this data.

### BACKGROUND ART

Electronic data (audio/video/images/infra-red etc.) contains sufficient attributes to identify the physical objects in a location. Humans can also be identified using the same methods. However, there is no current method to measure the objects and the emotions of the humans present in this data and make recommendations based on the brain activity of the users in a location and the optimal or preferred brain activity of an end user.

All current systems lack the ability to provide more detailed and intelligent recommendation to a user based on the user's preferences based on user's electroencephalogram (EEG) readings and the viewing angle of the user.

Accordingly, a need exists for a method, system and apparatus that builds a spatial model of a location (e.g. a room), identifies the EEG readings of the humans present in that location, the various audio and spatial interactions of the humans present in that location and recommends (or does not recommend) the location and the humans present in that location to an end user based on end user's brain activity model.

### SUMMARY OF THE INVENTION

In accordance with the present invention, there is provided a method, system and apparatus for building a recommendation system using the brain activity of users in an image or video, along with the spatial sensor data that includes depth information for the given image or video.

For instance, one embodiment of the present invention provides a method, system and apparatus for a device that is installed in a location and identifies all the objects in the location, and a device that is worn by all humans (or living things) in the location. The device in the location (such as a room) identifies the spatial features of the room such as depth and the various objects present. The device worn by the humans identifies the brain activity of the humans when they are interacting with the other humans at the given location and also the other objects at the location.

In one embodiment, the structural features of the location and the brain activity of participant users in captured in a file format that also has the image, video or audio of the room.

In one embodiment, brain wave readings along with other visual and audio data are recorded for a person over a period of time to build a personal machine learning model that represents the person.

In one embodiment, multiple avatars might be present in a single video or image.

In an embodiment, an internal avatar resident in the image might make recommendations to the user.

In another embodiment, the people present in the image or video can also have their internal brainwaves modeled and represented as avatars.

In an embodiment, the avatar might be represented using a visual image, an audio or both.

In one embodiment, an end user's brain activity is measured and modeled to create an avatar by subjecting the user to various images, audio, video and locations or situations.

In one embodiment, the angle of view of the end user looking at the image or the video is determined and a new image or video is recommended to the user.

In another embodiment, the new image or video is recommended using an internal avatar that may recommend using audio or visual cues.

In another embodiment the end user can communicate with the internal avatar by speaking to the internal avatar.

In one embodiment, the location and humans are captured in an image or a video.

In another embodiment, the objects and humans in a location as captured in video, audio or image are mapped to a conceptual taxonomy that can be used to highlight the important ideas (story) of the image.

In an embodiment, brain waves captured from real actors in a theater, movie or game setup are captured, merged to create a single model and relayed to viewers of the theater, movie or game.

In an embodiment, brain waves captured from real actors in a theater, movie or game setup are captured, to identify the variation of emotion each actor feels in a scene and this can be used to change the story line.

In an embodiment, movies are made using the spatial sensor for location information and the brain activity sensor for actor's emotions; the spatial and brain activity data is captured and merged to form an augmented reality, mixed reality or virtual reality movie.

In another embodiment, an end user's avatar explores various images, audio and videos in the virtual (or mixed or augmented) reality based image or video, and recommends the best results.

In another embodiment, an end user's depression or anxiety might be targeted so that it can be lowered by recommending the right stream of images, video and audio.

In another embodiment, the avatar might recommend user images based on user's Alzheimer's disease or memory loss problems.

In one embodiment, the brain activity of the humans in a movie is used to classify the human's acting on a given scale of bad to excellent. In another embodiment, this information is used to recommend the movie to the end user.

In another embodiment, a story is built taking into account end user's preferences, brain activity, viewing angle and the recommendations that it generates, where the new elements of the story are shown to the user based on the recommendations.

In one embodiment, a visual story telling is augmented with information of the actual emotional data that the actors are feeling. In another embodiment, this emotional data is used to quantify the story.

In one embodiment, retail and tourist places are used as location.

In another embodiment, standard machine and deep learning techniques and their combinations are used to create an avatar that uses the spatial and brain activity features to build a model.

### BRIEF DESCRIPTION OF THE DRAWINGS

The accompanying drawings, which are incorporated in and form a part of this specification, illustrate embodiments of the invention and, together with the description, serve to explain the principles of the invention.

FIG. 1 is a flowchart illustrating various processing parts used during building of a spatial and brain model.

FIG. 2 is a flowchart illustrating various processing parts for combining brain model.

FIG. 3 is a flowchart illustrating various processing parts for creating different story lines for theater, drama or movies.

FIG. 4 is a flowchart of steps performed for capturing long term human behavior.

FIG. 5 is a flowchart of steps performed for adding brain model to augmented, virtual or mixed reality images or video.

FIG. 6 is a block diagram of an embodiment of an exemplary computer system used in accordance with one embodiment of the present invention.

### DETAILED DESCRIPTION OF THE INVENTION

Reference will now be made in detail to the preferred embodiments of the invention, examples of which are illustrated in the accompanying drawings. While the invention will be described in conjunction with the preferred embodiments, it will be understood that they are not intended to limit the invention to these embodiments.

On the contrary, the invention is intended to cover alternatives, modifications and equivalents, which may be included within the spirit and scope of the invention as defined by the appended claims. Furthermore, in the following detailed description of the present invention, numerous specific details are set forth in order to provide a thorough understanding of the present invention. However, it will be obvious to one of ordinary skill in the art that the present invention may be practiced without these specific details. In other instances, well-known methods, procedures, components and circuits have not been described in detail as not to unnecessarily obscure aspects of the present invention.

### Notation and Nomenclature

Some portions of the detailed descriptions, which follow, are presented in terms of procedures, logic blocks, processing and other symbolic representations of operations on data bits within a computer system or electronic computing device. These descriptions and representations are the means used by those skilled in the data processing arts to most effectively convey the substance of their work to others skilled in the art. A procedure, logic block, process, etc., is herein, in generally, conceived to be a self-sequence of steps or instructions leading to a desired result. The steps are those requiring physical manipulations of physical quantities. Usually, though not necessarily, these physical manipulations take the form of electrical or magnetic signals capable of being stored, transferred, combined, compared, and otherwise manipulated in a computer system or similar electronic computing device. For reasons of convenience, and with reference to common usage, these signals are referred to as bits, values, elements, symbols, characters, terms, numbers, or the like with reference to the present invention.

It should be borne in mind, however, that all of these terms are to be interpreted as referencing physical manipulations and quantities and are merely convenient labels and are to be interpreted further in view of terms commonly used in the art. Unless specifically stated otherwise as apparent from the following discussions, it is understood that throughout discussions of the present invention, discussions utilizing terms such as “generating” or “modifying” or “retrieving” or the like refer to the action and processes of a computer system, or similar electronic computing device that manipulates and transforms data. For example, the data is represented as physical (electronic) quantities within the computer system's registers and memories and is transformed into other data similarly represented as physical quantities within the computer system memories or registers or other such information storage, transmission, or display devices.

### Exemplary System in Accordance with Embodiments of the Present Invention

### The Spatial and Brain Model Builder

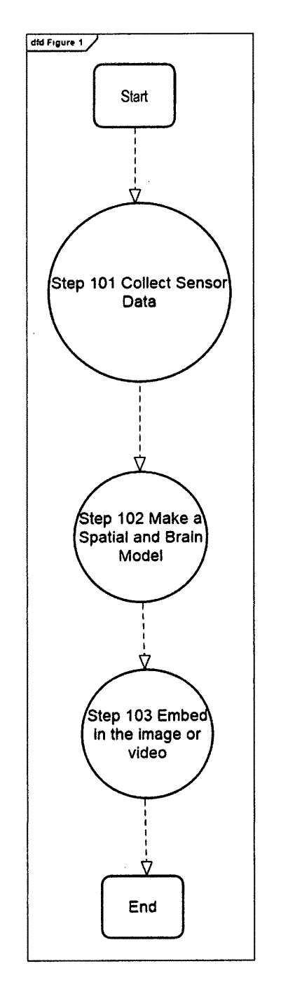

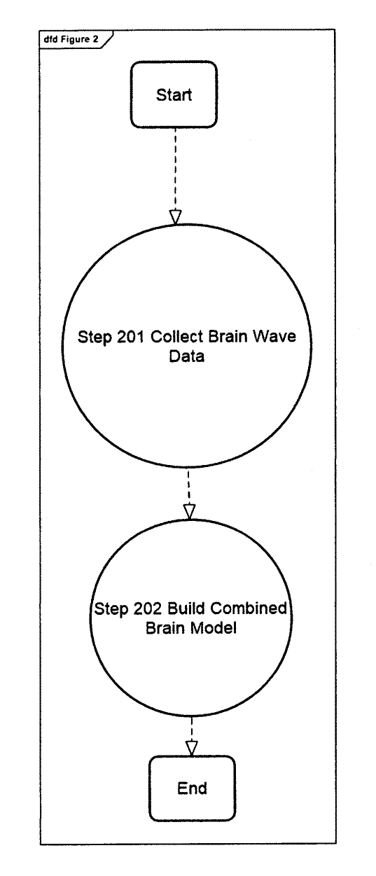

FIG. 1 consists of the steps performed by the spatial and emotion model builder. The sensor data is collected in Step 101 and the model built in Step 102. The model is embedded with the image data in Step 103.

### Combining Brain Models

FIG. 2 consists of the steps performed by the brain wave combiner. The combined brain model serves to augment the visual and audio recommendations.

The sensor data is collected in Step 201 from multiple users, the model is built in Step 202 using machine learning/neural methods. The individual user's models are built by providing them an external stimulus. The external stimulus can be in the form of sound, music, images, video and so on. The combined model is used to recommend images and music that creates a stimulus in the target subject.

### Combining Brain Models to Create Alternate Story Lines

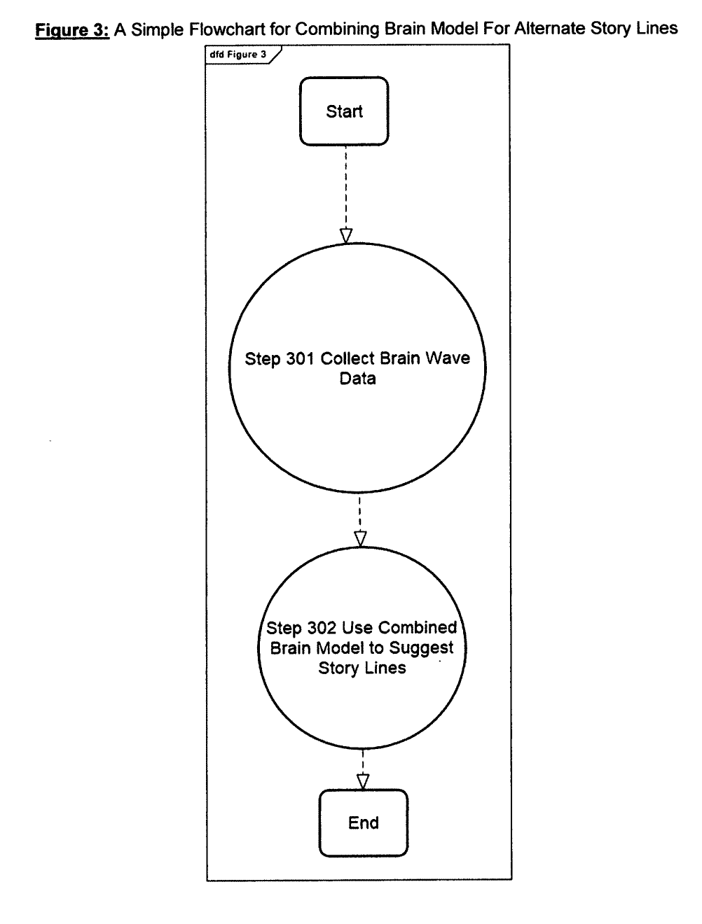

FIG. 3 consists of the steps performed to recommend story lines in an augmented reality set up. The combined brain model created when used by actors in a game, theatrical or movie setup, can be used to improve scenes in a drama, movie or game.

The sensor data collected in Step 301 from multiple actors is used to build the model is built in Step 302 using machine learning/neural methods. The built model is used to suggest new story line twists based on the actor's interpretations of the story as per their emotions in Step 302.

### Using Long Term Brain Models to Capture Personal Emotional Behavior

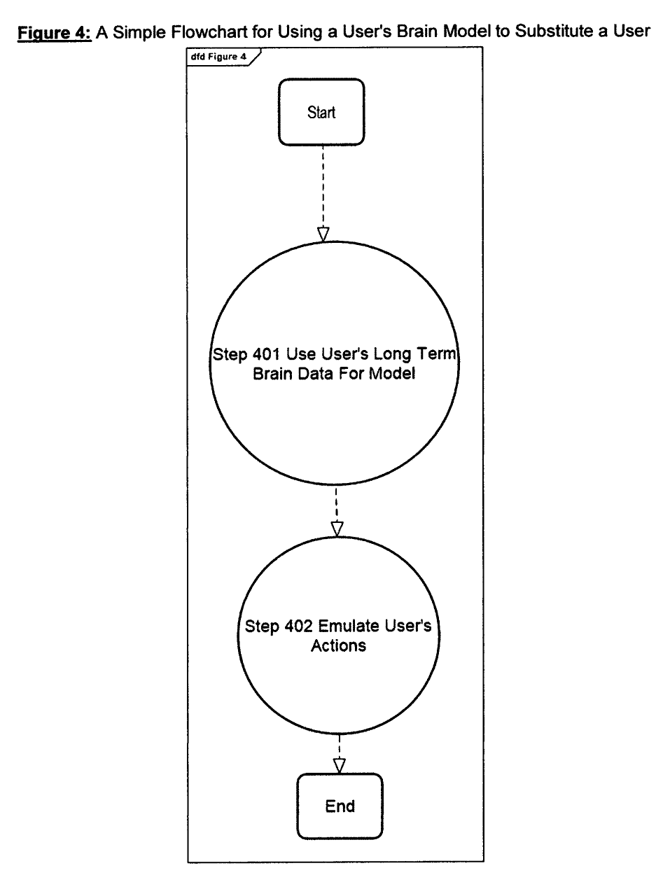

FIG. 4 consists of the steps performed to capture emotional behavior of a person when exposed to external stimulation over a long period of time. The emotional behavior is stored in a model that represents the specific user in Step 401.

The built model is used to substitute the user's actions in the absence of the user in Step 402.

### Combining Brain Models with Augmented, Virtual or Mixed Reality

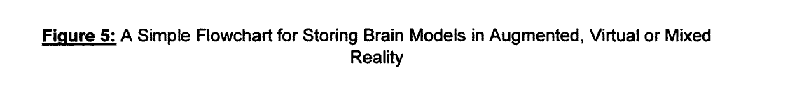

FIG. 5 consists of the steps performed to combine brain models with augmented, virtual or mixed reality environments. Brain models are embedded as scene specific objects in these environments in Step 501.

The embedded model responds when the external user provides a stimulus to the model by either clicking on it, viewing it, talking to it or exploring a video or audio input within a scene in Step 502.

### Exemplary Operations in Accordance with Embodiments of the Present Invention

FIGS. 1-5 are flowcharts of computer-implemented steps performed in accordance with one embodiment of the present invention for providing a method, system and apparatus for Brain Wave Based Recommendations.

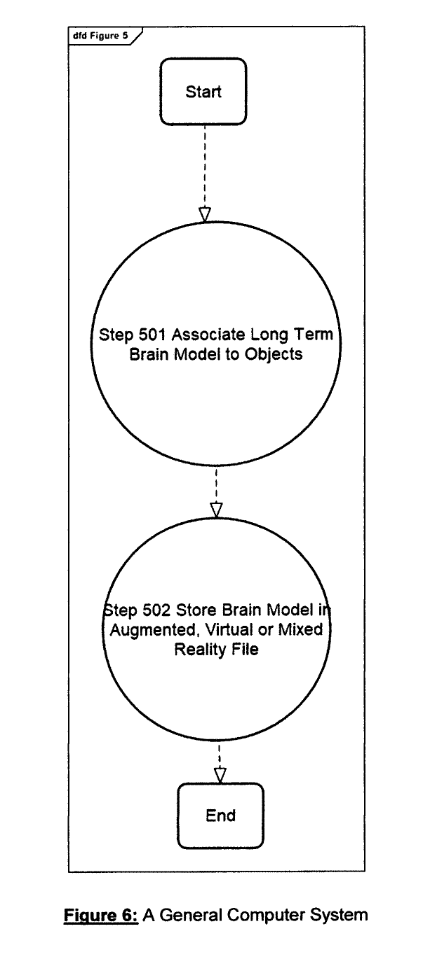

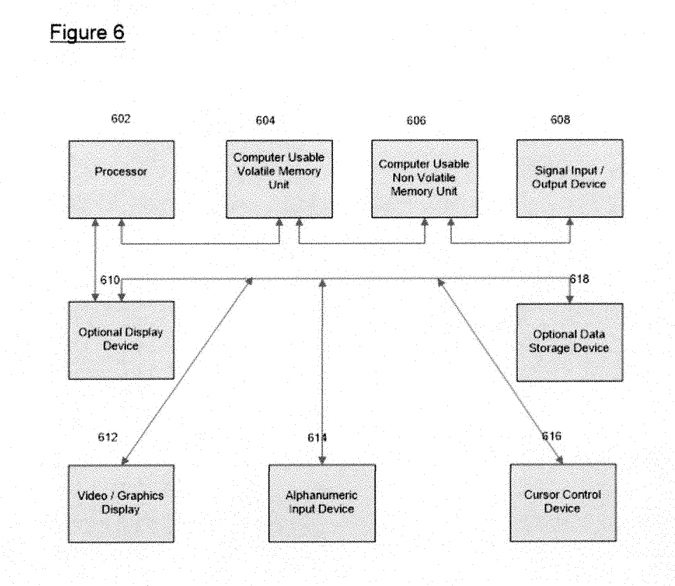

The flowcharts include processes of the present invention, which, in one embodiment, are carried out by processors and electrical components under the control of computer readable and computer executable instructions. The computer readable and computer executable instructions reside, for example, in data storage features such as computer usable volatile memory (for example: 604 and 606 described herein with reference to FIG. 6). However, computer readable and computer executable instructions may reside in any type of computer readable medium. Although specific steps are disclosed in the flowcharts, such steps are exemplary. That is, the present invention is well suited to performing various steps or variations of the steps recited in FIG. 1-5. Within the present embodiment, it should be appreciated that the steps of the flowcharts may be performed by software, by hardware or by any combination of software and hardware.

### Automatic Generation of Spatial and Brain Model and it's Usage

The method, system and apparatus of the present invention provide for gathering sensor data, identifying physical objects in a spatial location, detecting emotions and personal preferences of humans present in the location and building a machine learning model is disclosed.

According to one embodiment, an end user's avatar is created that serves as a client to this system and explores various images and videos on its own to recommend.

In another embodiment, an end user is shown various videos, audio and images and the user's brain activity is recorded to create machine learning model

### Exemplary Hardware in Accordance with Embodiments of the Present Invention

FIG. 6 is a block diagram of an embodiment of an exemplary computer system 600 used in accordance with the present invention. It should be appreciated that the system 600 is not strictly limited to be a computer system. As such, system 600 of the present embodiment is well suited to be any type of computing device (for example: server computer, portable computing device, mobile device, embedded computer system, etc.). Within the following discussions of the present invention, certain processes and steps are discussed that are realized, in one embodiment, as a series of instructions (for example: software program) that reside within computer readable memory units of computer system 600 and executed by a processor(s) of system 600. When executed, the instructions cause computer 600 to perform specific actions and exhibit specific behavior that is described in detail below.

Computer system 600 of FIG. 6 comprises an address/data bus 610 for communicating information, one or more central processors 602 couples with bus 610 for processing information and instructions. Central processing unit 602 may be a microprocessor or any other type of processor. The computer 600 also includes data storage features such as a computer usable volatile memory unit 604 (for example: random access memory, static RAM, dynamic RAM, etc.) coupled with bus 602, a computer usable non-volatile memory unit 606 (for example: read only memory, programmable ROM, EEPROM, etc.) coupled with bus 610 for storing static information and instructions for processor(s) 602. System 600 also includes one or more signal generating and receiving devices 608 coupled with bus 610 for enabling system 600 to interface with other electronic devices. The communication interface(s) 608 of the present embodiment may include wired and/or wireless communication technology. For example, in one embodiment of the present invention, the communication interface 608 is a serial communication port, but could also alternatively be any of a number of well known communication standards and protocols, for example: Universal Serial Bus (USB), Ethernet, FireWire (IEEE 1394), parallel, small computer system interface (SCS), infrared (IR) communication, Bluetooth wireless communication, broadband, and the like.

Optionally, computer system 600 can include an alphanumeric input device 614 including alphanumeric and function keys coupled to the bus 610 for communicating information and command selections to the central processor(s) 602. The computer 600 can include an optional cursor control or cursor-directing device 616 coupled to the bus 610 for communicating user input information and command selections to the central processor(s) 602. The system 600 can also include a computer usable mass data storage device 618 such as a magnetic or optional disk and disk drive (for example: hard drive or floppy diskette) coupled with bus 610 for storing information and instructions. An optional display device 612 is coupled to bus 610 of system 600 for displaying video and/or graphics.

As noted above with reference to exemplary embodiments thereof, the present invention provides a method, system and apparatus generating a recommendation based on spatial and emotions' models based on sensor data.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of invention and its practical application, to thereby enable others skilled in the art to best utilize the invention and various embodiments with various modifications as are suited to the particular use contemplated. It is intended that the scope of the invention to be defined by the claims appended hereto and their equivalents.

1. A method comprising:
processing sensor data, and objects identified in the sensor data to create a model that connects the identified objects to brain waves
whereby said model is used to create a story in augmented, virtual or mixed reality using recommendations.

2. The method of claim 1, wherein the sensor data object identification comprises of identification of objects present in a location using physical attributes not limited to image, sound, movement, angle of view, brain activity and heat

3. A story creation system comprising:
1. means adapted for collecting heterogeneous sensor data,
2. means for identifying physical objects in the sensor data,
3. means adapted for building a machine learning model that connects the physical objects to the brain waves
whereby said model is used to create a story in augmented, virtual or mixed reality using recommendations.

4. The system of claim 3, wherein the sensor data object identification comprises of identification of objects present in a location using physical attributes not limited to image, sound, movement, angle of view, brain activity and heat.

5. A non-transitory computer readable medium of instructions comprising:
instructions for processing sensor data, and objects identified in the sensor data to create a model that connects the identified objects to brain waves
whereby said model is used to create a story in augmented, virtual or mixed reality using recommendations.

6. The non-transitory computer readable medium of instructions of claim 5, the sensor data object identification comprises of identification of objects present in a location using physical attributes not limited to image, sound, movement, angle of view, brain activity and heat.

7. The model of claim 1, wherein the machine learning model is built using the combined brain waves of multiple people.

8. The model of claim 1, wherein the model is embedded as an Avatar in the media file.

9. The story of claim 1, wherein the story is updated based on the brain waves of the actors in the story.

10. The story of claim 1, wherein the story is used for various brain related diseases not limited to depression and Alzheimer's disease.

11. The story of claim 1, wherein the actors in the story are rated based on the brain waves.

12. The system of claim 3, wherein the machine learning model comprises of combined brain waves of multiple people.

13. The system of claim 3, wherein the model is embedded as an Avatar in the media file.

14. The system of claim 3, wherein the story is updated based on the brain waves of the actors in the story.

15. The system of claim 3, wherein the story is used for various brain related diseases not limited to depression and Alzheimer's disease.

16. The system of claim 3, wherein the actors in the story are rated based on the brain waves.

17. The non-transitory computer readable medium of instructions of claim 5, wherein the machine learning model comprises of combined brain waves of multiple people.

18. The non-transitory computer readable medium of instructions of claim 5, wherein the model is embedded as an Avatar in the media file.

19. The non-transitory computer readable medium of instructions of claim 5, wherein the story is updated based on the brain waves of the actors in the story.

20. The non-transitory computer readable medium of instructions of claim 5, wherein the story is used for various brain related diseases not limited to depression and Alzheimer's disease.
